(19) United States
(12) Patent Application Publication
(10) Pub. No.: US 2005/0149016 A1
(43) Pub. Date: Jul. 7, 2005

(54) Laparoscopic device and method of female sterilization

(75) Inventor: Syed Rizvi, Bakersfield, CA (US)

Correspondence Address: JEFFREY FURR, 253 N. MAIN STREET, JOHNSTOWN, OH, 43031, US

(73) Assignee: CENTUM RESEARCH LLC, Bakersfiled, CA (US)

(21) Appl. No.: 10/707,637

(22) Filed: Dec. 29, 2003

(51) Int. Cl. A61B 18/18

(52) U.S. Cl. 606/51

(57) ABSTRACT

A laparoscopy instrument and method which allows simultaneous cauterization, dissection and removal of body tissue. Mechanisms for grasping, holding, manipulating, cauterizing, dissecting and removing the tissue are provided in one implement allowing easy, safe and quick performance of the procedure.

### BACKGROUND OF INVENTION

1. Field of the Invention

This invention relates to a laparoscopy instrument and method, which allows simultaneous manipulation, cauterization, dissection and removal of the body tissue.

2. Background of the Invention

Tubal sterilization is a permanent method of birth control in which a portion of the fallopian tube is interrupted and either tied, cut, clipped, blocked, cauterized or removed using laparoscopic, hysteroscopic or open methods. Tubal sterilization is the most commonly used method of birth control. Tubal sterilization also is a highly effective method for women choosing to permanently terminate their reproductive ability.

Rioux (U.S. Pat. No. 3,938,527) described a laparoscopic electrocautery instrument for tubal cauterization.

Yoon (U.S. Pat. No. 3,967,625) described a method for sterilizing the female by tubal ligation comprising the use of a ring and an applicator device.

Polk (U.S. Pat. No. 4,226,239) described a surgical ligating instrument for tubal ligation by the application of two or more elastic rings.

Yoon (U.S. Pat. No. 4,374,523) describes a two cylinder ring applicator device and method for applying an elastic occluding ring on fallopian tubes.

Young (U.S. Pat. No. 5,300,081) describes an apparatus for applying surgical clips on the fallopian tubes.

Koros (U.S. Pat. No. 5,352,235) describes a monopolar laparoscopic instrument with scissor like blades to cut the tissue.

McQuilkin (U.S. Pat. No. 5,575,802) described a clip suitable for sexual sterilization.

Koros (U.S. Pat. No. 5,578,052) describes insulated laparoscopic grasper with removable shaft.

Whitfield (U.S. Pat. No. 5,833,696) described a surgical clip applying instrument.

Jones (U.S. Pat. No. 6,112,747) describes a method of female sterilization with the application of laser energy through the hysteroscope.

Davis (U.S. Pat. No. 6,241,740) describes an instrument and method for ligating with a clip and cutting the tissue.

Harrington (U.S. Pat. No. 6,309,384) describes a method, which involves thermally damaging the lining of the utero-tubal junction followed by placement of a foam plug.

Technical advances, particularly the use of the laparoscope, have significantly affected the setting in which tubal sterilization procedures can be performed and the trend toward sterilization. Laparoscopic method of sterilization continues to increase in usage. Laparoscopic tubal sterilization instrumentation and methods in prior art do not allow the simultaneous manipulation, bipolar cauterization, clean symmetrical dissection and the removal of a portion of fallopian tube. Laparoscopic instrumentation in prior art are not ergonomically efficient to provide for maximum operating capability needed to perform all the necessary steps of the operation. Lack of instrumentation and methods of performing simultaneous manipulation, bipolar cauterization, clean symmetrical dissection and removal of a portion of the fallopian tube with ergonomic efficiency make it very difficult for surgeons to perform such an operation using minimally invasive techniques and without exposing patients to risks of serious complications.

During unipolar cautery, which can be performed with or without the laparoscopic instrument, patient''s body is part of the circuit. This method has been associated with dangerous and fatal consequences due to injuries to other organs.

The bipolar cautery technique is the safest and simplest to perform. The poles of the tip of the instrument conduct the electricity between them, with no current flow beyond the instrument tip, so the patient is not part of the circuit eliminating the risk of injury to other organs.

Methods of tubal sterilization where a portion of the tube is not cut and removed can fail due to the recanalization of the fallopian tube. These operations can also cause life threatening ectopic pregnancies in the future. In view of this background there is a need for an easy, safe, effective, minimally invasive and fast method of tubal sterilization. Said device and method provide for such a procedure.

### SUMMARY OF INVENTION

A laparoscopy instrument and method, which allows simultaneous manipulation, cauterization, dissection and removal of the body tissue. Distal end of the instrument allows lifting and grasping the fallopian tube and cauterizing/cutting it. The ring shape jaw of the distal end of the instrument allows cauterizing the tissue. The cup shape part of the distal end of the instrument allows dissecting and removing the portion of the tube in it''s entirety in one step. The instrument can be used on any part of the body where simultaneous manipulation, cauterization and/or hemostasis and tissue dissection and/or removal are desired. Handle of the device and attachments are used to open/close the jaws, manipulate the device and the tissue and to operate the cautery/cutting mechanisms. Additional embodiment in the handle allows the use of middle finger to operate the electrocautery providing maximum operating capability without the need for repositioning the hand or the fingers.

The sources of energy used include but are not limited to electrical, ultrasonic, laser or radio energy.

Instrument can be a modular laparoscopic instrument providing surgical efficiency, convenience, precision, durability and cost effectiveness by replacing only the disposable tip and reusing the non-disposable hand piece.

### BRIEF DESCRIPTION OF DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 displays an overview of the device comprising;

FIG. 2 displays the proximal portion (handle) comprising; and

FIG. 3 displays the distal part (tip) comprising.

### DETAILED DESCRIPTION

Drawings show the said instrument. Dimensions of the preferred embodiments in the drawings are consistent with the standard dimensions of the laparoscopic instruments currently in use. These dimensions can vary as ever changing technology allows for miniaturization of laparoscopic devices.

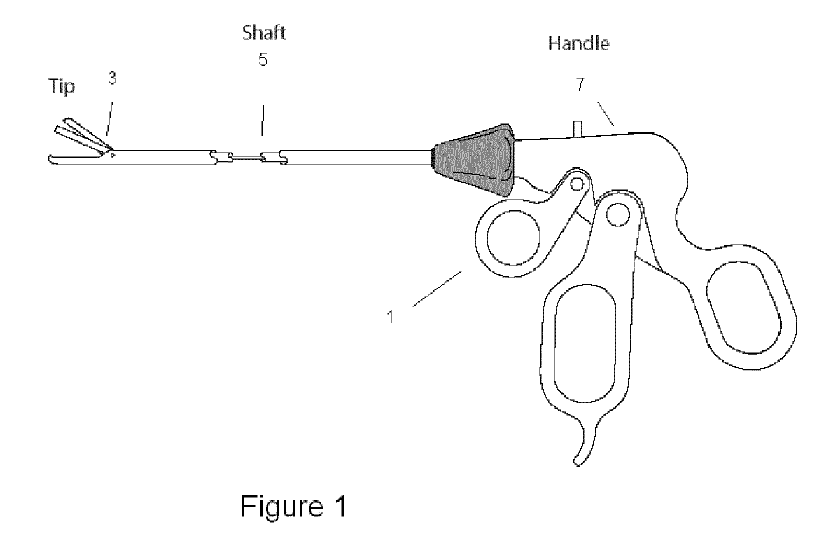

FIG. 1 shows the overview of the device 1. The Handle 7 of the device 1 is in the proximal portion. The Shaft 5 of the device 1 is in the middle portion. The Tip 3 of the device 1 is in the distal portion.

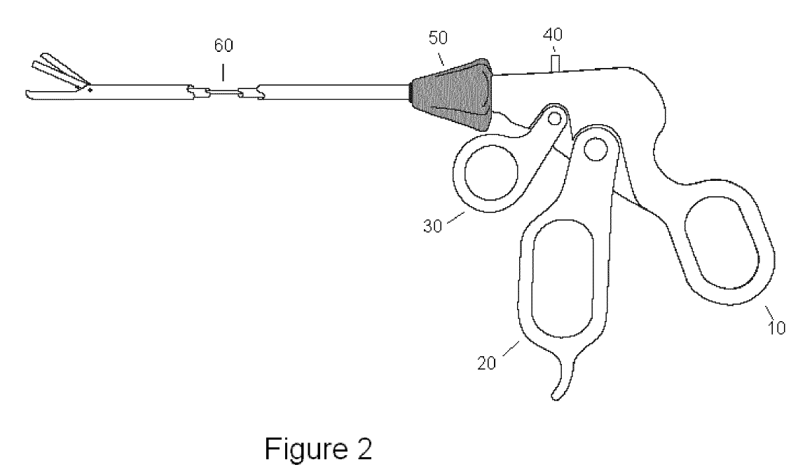

FIG. 2 shows the proximal portion (handle) of the device 1. The slightly round shape fixed part 10 of the handle for the placement of the thumb. This part is approximately 4.5 centimeters long and 3 centimeters wide and is attached to the fixed portion of the handle, which is approximately 7 centimeters long.

The elongated movable part 20 of the handle 7 for positioning 4^{th} and 5^{th} fingers and is connected to the cutting jaw G in the distal portion of the device. Forward movement of this part opens the cutting jaw and backward movement closes the cutting jaw. This part is approximately 10 centimeters long and 3 centimeters wide.

The round shape movable embodiment 30 in the handle 3 connected to the cautery ring 80 in the distal portion of the device. Forward movement of this embodiment opens and the backward movement closes the cautery ring. This embodiment can be used with the middle finger. This part is approximately 2.5 centimeters wide and 4 centimeters long.

The rotator attachment 50 to rotate the shaft 60 of the device 1 with the index finger and is approximately 2.5 centimeters long.

The bipolar cautery attachment 40 is for bipolar cautery.

The connector 60 part of the shaft connecting the proximal (handle) and the distal (tip) portions of the device is approximately 31 centimeters long.

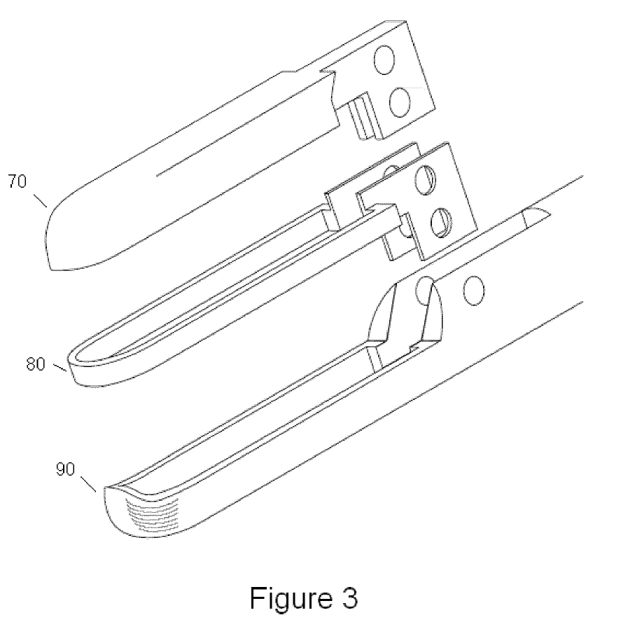

FIG. 3 shows the distal part (tip). The cup shape cutting jaw 70 is connected to the elongated movable part 20 of the handle 7 via the shaft 5. Forward movement of the elongated movable part 20 in the handle 7 opens this jaw 70 and backward movement closes this jaw 70. The cutting part of the jaw 70 is approximately 1.5 centimeters long.

The cautery ring 80 is connected to the embodiment 30 of the handle 7 via the shaft 5. Forward movement of the embodiment 30 opens and the backward movement closes this cautery ring 80. Total length of this part is approximately 2.2 centimeters.

The fixed jaw 90 connected to the handle 7 via the shaft 5 and the cautery and is approximately 2.2 centimeters long.

The preferred diameter of the shaft 5 and the tip 3 is 7 millimeters. Fixed lower jaw 90 is 7 millimeters wide. Ring shape cautery jaw 80 is 7 millimeters wide and the cup shape cutting jaw 70 is 5 millimeters wide.

Difference in the width of the ring shape cautery jaw 80 and cup shape cutting jaw 70 allows for at least one millimeter margin between the cauterized tissue and the dissected edge providing for the added safety by reducing the risk of complications.

### OPERATION OF THE INVENTION

The patient is given general anesthesia and is placed in low stirrups. Vaginal retractor is placed and anterior os of the cervix is grasped with a tenaculum. A uterine manipulator is placed to manipulate the uterus. A small incision is made in the inferior fold of the umbilicus. And a verres canula is then inserted into the peritoneal cavity for carbon dioxide insufflation. A sufficient quantity of carbon dioxide is insufflated while the patient is in a steep Trendelenburg position. A trocar is then introduced into the abdominal cavity and the laparoscope inserted through the sleeve.

A secondary mini incision is made in the midline at the bikini line and a 7 millimeter trocar is introduced under direct vision. The said device is inserted through the trocar.

Pelvic organs including right and left fallopian tubes are visualized. Tubes can be manipulated and mobilized into a desired position using the said device. Uterine manipulator is used to manipulate the uterus as needed. Jaws of the said device are opened using embodiments 20 and 30. Middle portion of the one fallopian tube is grasped assuring that the entire tube is in the jaws of the device 1. The cauterizing jaw is closed by using the embodiment 30 on the handle.

The surgeon then actuates the bipolar cautery and cauterizes the tube. If desired the tube can be cauterized at two or more locations for added benefits. The surgeon now engages the cutting mechanism by using the embodiment 20 on the handle 7 until the tube is completely dissected. This traps the dissected portion of the tube into the jaws. The cautery jaw is then opened slightly, instrument is pulled back and the jaw is closed. The specimen is removed by pulling the device back and bringing it out of the peritoneal cavity. The specimen is released by opening the jaws and sent for histological evaluation if so desired. Same procedure is repeated on the contralateral side.

After the procedure laparoscope is removed. Peritoneal cavity is deflated. Trocars are removed and mini incisions are closed. Vaginal instruments are removed.

It is to be understood that both the foregoing general description and the following detailed description are exemplary and explanatory but are not restrictive of the invention.

The invention in its broader aspects is not limited to the specific details shown and described, and departures may be made from such details without departing from the principles of the invention and without sacrificing its chief advantages.

1. A laparoscopy instrument comprising;
a shaft connecting a proximal end and a distal end;
a handle on said proximal end, said handle attached to a power source for bipolar electrocautery with said handle housing embodiments to engage cautery means and cutting means and a shaft rotation means;
a tip on said distal end, with said tip housing one fixed and two movable jaws, said fixed jaw and one movable jaw connected to said power source for bipolar cautery via said shaft, the other movable jaw is a cup shape cutting jaw connected to said handle via said shaft.

2. An ergonomically efficient embodiment in instrument as in claim 1 allowing surgeon to use middle finger for additional step during a surgical procedure without removing or repositioning the fingers.

3. A method using the device claimed in claim 1.

4. A method as in claim 3 having the simultaneous manipulation, cauterization, dissection and removal of body tissue.

5. A method as in claim 3 having the simultaneous cauterization, dissection and removal of the parts of the fallopian tubes.

6. A method as in claim 3 having the simultaneous cauterization, dissection and removal of the parts of the fallopian tubes in it's entirety.

7. A method as in claim 3 performing female sterilization.

8. A laparoscopy instrument comprising;
a shaft connecting a proximal end and a distal end;
a handle on said proximal end, said handle attached to a power source for bipolar electrocautery with said handle housing embodiments to engage cautery means and a shaft rotation means;
a tip on said distal end, with said tip housing one fixed and two movable jaws, said fixed jaw and one movable jaw connected to said power source for bipolar cautery via said shaft, the other movable jaw is a cup shape cutting jaw connected to said handle via said shaft.

9. An ergonomically efficient embodiment in instrument as in claim 1 allowing surgeon to use middle finger for additional step during a surgical procedure without removing or repositioning the fingers.

10. A method using the device claimed in claim 8.

11. A method as in claim 10 having the simultaneous manipulation, cauterization, dissection and removal of body tissue.

12. A method as in claim 10 having the simultaneous cauterization, dissection and removal of the parts of the fallopian tubes.

13. A method as in claim 10 having the simultaneous cauterization, dissection and removal of the parts of the fallopian tubes in it's entirety.

14. A method as in claim 10 performing female sterilization.
